(19) United States
(12) Patent Application Publication
(10) Pub. No.: US 2018/0125044 A1
(43) Pub. Date: May 10, 2018

(54) Method for Cultivating a Vegetable worms Having a Function of a Natural Preserved Agent and a Freshness Keeping Agent and an Extract from the Same and a Natural Complex Composition From the Same

(71) Applicant: BST Inc., Gyeonggi-do (KR)

(72) Inventors: Ho Lee, Gyeonggi-do (KR); Dong-Myong Kim, Seoul (KR); Chul Kim, Gyeonggi-do (KR); Kyung-Hyun Im, Gyeonggi-do (KR); Myung-Kuk Shim, Seoul (KR)

(21) Appl. No.: 15/346,470

(22) Filed: Nov. 8, 2016

(51) Int. Cl. A01K 67/033

(52) U.S. Cl. CPC A01K 67/033; A01K 2267/00; A01K 2227/703

(57) ABSTRACT

The present invention relates to a method for cultivating vegetable worms having a function of a natural preserved agent and freshness keeping agent. The method comprises obtaining a mycelium by inoculating a cordyceps militaris or a cordyceps nutans to a liquid medium, a solid medium or a worm body; and forming a fruit body from the mycelium by cultivating a solid medium or a worm body.

### BACKGROUND OF THE INVENTION

### 1. Field of the Invention

The present invention relates to a method for cultivating vegetable worms having a function of a natural preserved agent and freshness keeping agent, an extract from the same and a natural complex composition from the same, in particular the method for cultivating the vegetable worms at a culture medium and fermenting the same for improving an effectiveness of a cordycepin, and the agent containing a component of a green tea, a component of a garlic, a component of a coconut, a component of a grape and the like for antibiotic preservation or a freshness preservation, and the complex composition containing a natural polymer for the same.

2. Description of the Related Art

Food or a vegetable may be treated by heating, an ultra-high pressure, radiating ray or light pulse, or an additive such as synthetic preservation agent containing a benzoic acid, sorbic acid, a chlorine agent, a nitrite, a sodium sulfite or the like may be added in the food or the vegetable. But the method for preserving the food or the vegetable by the known art has a problem that the physical treatment or the addition of the synthetic preserving agent may destruct nutrition or make a side-effect by accumulation within a part of body. Hence, it is a major issue that the use of the synthetic preserving agent be diminished and a substitute agent for the synthetic preserving agent such as a natural antibiotic material be developed. For example, a substance such as sodium, vinegar, protein extracted from plant, nisin, e-polylysine, natamycin, chitosan or organic acid may be utilized as a natural antibiotic agent. But, the known natural antibiotic agent have a problem that the agent have certain taste, smell or stimulating property, weak antibiotic property or narrow antibiotic range. Therefore, it is necessary for an antibiotic agent having a property equal or better that of the synthetic preserving agent. In addition, it is advantageous that the agent may be utilized as an agent for freshness.

### PURPOSE OF THE INVENTION

A purpose of the present invention is to provide a method for cultivating vegetable worms in a medium.

Other purpose of the present invention is to provide an extract from vegetable worms cultivated in a synthetic medium and utilized as a natural antibiotic preserving agent and a natural agent for preserving freshness having an equal to or better than that of a synthetic agent.

Another purpose of the present invention is a complex composition for substituting a synthetic agent for preserving freshness of food or vegetable and for antibiotic effect.

### SUMMARY OF THE INVENTION

According to an aspect of the present invention, a method for cultivating vegetable warms in a medium comprises obtaining a mycelium by inoculating a cordyceps militaris or a cordyceps nutans to a liquid medium, a solid medium or a worm body;

and forming a fruit body from the mycelium by cultivating a solid medium or a worm body, wherein the solid medium for cultivating the fruit body is formed by a mixture of a corn material and the liquid medium or a mixture of a rice material, a silkworm pupa material and the liquid medium; the solid medium is under a condition of 50 to 70 wt % moisture content, 80 to 95% relative humidity, 15 to 25° C. atmosphere temperature and 700 to 1,200 lux luminous intensity; the liquid for the liquid medium contains MgSO₄.7H₂O, KH₂.PO₄, Ka₂.HPO₄, a yeast extract, a peptone and a dextrose or contains a yeast extract, a dextrose, an ebios and a Hyponex; the solid medium for the mycelium is formed from the liquid medium and at least one selected from a group consisting of a rice material, a potato material, a corn material and a silkworm pupa material.

According to other aspect of the present invention, the method for cultivating vegetable worms further comprises fermenting an extract from the mycelium or the fruit body by a lactic acid bacterium.

According to another aspect of the present invention, a natural complex composition is extracted from a mycelium or a fruit body cultivated in a liquid medium, a solid medium or a worm body; and comprises extracted components consisting of 2.0 to 8.0 wt % water; 2.0 to 3.5 wt % crude fat; 30 to 50 wt % raw protein; 5.0 to 8.0 wt % raw ash; and additives comprising a cellulose, a pectin, a carrageenan, a starch, a protein, an enzyme or a chitosan, wherein the composition is made in a form to be sprayed or to be added onto a vegetable or a food.

According to still aspect of the present invention, a complex composition comprises components extracted from vegetable worms; a base material containing 2.0 to 8.0 wt %, 2.0 to 3.5 wt % raw fat, 30 to 50 wt % raw protein and 5.0 to 8.0 wt % raw ash; and additives containing a green tea, a garlic, a coconut, a grape or an oriental medicine stuff.

### BRIEF DESCRIPTION OF THE DRAWINGS

The above and other aspects, features and advantages of certain exemplary embodiments of the present invention will be more apparent from the following detailed description taken in conjunction with the accompanying drawings, in which:

FIG. 1a to 1i shows an effect of an antibiotic preserving agent made of an extract according to one embodiment of the present invention. Referring to FIG. 1a to 1i, the control is an antibiotic preserving agent extracted from a vegetable worms that grow in nature; the storage time is per a day; A, B and C represent a hamburger patty, a fish cake and a noodle, respectively.

FIG. 2a to FIG. 2c shows a pH effect result treated by an extract according to embodiment of the present invention. Referring to FIG.2a to FIG. 2c, the control is an antibiotic preserving agent extracted from vegetable worms that grow in nature, and; the storage time is per a day; A, B and C represent a hamburger patty, a fish cake and a noodle, respectively.

FIG. 3a to FIG. 3c shows an acid changing result treated by an extract according to embodiment of the present invention. Referring to FIG. 2a to FIG. 2c, the control is an antibiotic preserving agent extracted from vegetable worms that grow in nature, and; the storage time is per a day; A, B and C represent a hamburger patty, a fish cake and a noodle, respectively.

FIG. 4a to FIG. 4c shows a sensing test result treated by an extract according to embodiment of the present invention. Referring to FIG. 2a to FIG. 2c, the control is an antibiotic preserving agent extracted from vegetable worms that grow in nature; the storage time is per a day; the periods of storage are 1, 2, 3 and 4 day; and the temperature of the storage is 12° C.

FIGS. 5a to 5d shows a toxic result treated by an extract according to embodiment of the present invention, and FIG.5a to 5d represents the result treated at 1% concentration to A375 cell and HaCat cell, respectively.

FIGS. 6a to 6c and FIGS. 7a to 7e show freshness preserving result of treated by an extract according to embodiment of the present invention. FIG. 6a represents an evaluating result related to a capability of a keeping humidity, and the result shows result by a coating agent available commercially and by the extract according to the present invention. FIGS. 7a to 7c shows the result in 5 days, 10 days and 20 days after being inoculated, and FIG. 7d and FIG. 7e show result in 1 to 6 days and 16 to 22 days after being inoculated.

### DETAILED DESCRIPTION OF EXEMPLARY EMBODIMENTS

Exemplary embodiments of the present invention will be described herein below with reference to the accompanying drawings. In the following description, well-known functions or constructions are not described in detail since they would obscure the invention in unnecessary detail. Also, the terms used herein are defined according to the functions of the present invention. Thus, the terms may vary depending on user's or operator's intension and usage. That is, the terms used herein must be understood based on the descriptions made herein.

A method for cultivating vegetable worms according to the present invention comprises obtaining a mycelium by inoculating a cordyceps militaris or a cordyceps nutans to a liquid medium, a solid medium or a worm body; and forming a fruit body from the mycelium by cultivating a solid medium or a worm body, wherein the solid medium for cultivating the fruit body is formed by a mixture of a corn material and the liquid medium or a mixture of a rice material, a silkworm pupa material and the liquid medium; the solid medium is under a condition of 50 to 70 wt % moisture content, 80 to 95% relative humidity, 15 to 25° C. atmosphere temperature and 700 to 1,200 lux luminous intensity; the liquid for the liquid medium contains MgSO₄.7H₂O, KH₂.PO₄, K₂.HPO₄, a yeast extract, a peptone and a dextrose or contains a yeast extract, a dextrose, an ebios and a Hyponex; the solid medium for the mycelium is formed from the liquid medium and at least one selected from a group consisting of a rice material, a potato material, a corn material and a silkworm pupa material.

The vegetable worms cultivated at the medium may be a form of a mycelium or a fruit body, and the mycelium or the fruit body may be separated from the medium. And effective components may be extracted from the separated mycelium or the fruit body of the vegetable worms. The extract obtained from the mycelium or the fruit body through fermentation has function of an antibiotic activity, preservation, freshness preservation or anti-oxidation on being added into food, medical produce, cosmetic, fiber product or a daily supply. The mycelium or the fruit body of the vegetable worms cultivated at the medium may have identical components, and are found that it contains a cordycepin component for antibiotic preservation or keeping freshness. As the result, it has been found that the extract according to the present invention has the amount of effective components equal to or better capability than vegetable worms in nature and, hence, replaces a synthetic agent.

The detailed description is shown in following.

1. Cultivation of Mycelium

The mycelium can be obtained by being cultivated at a liquid medium, a solid medium or a worm body, and the media may be made in various ways.

1) Cultivation of Mycelium at the Liquid Medium

A cordyceps militaris or cordyceps nutans bacterium was prepared, inoculated at the 7 kinds of 20 M [symbol] liquid media, respectively and, then, cultivated for 7 days. The condition for cultivating is 24˜25° C. temperature, 1.0±0.5 vvm airflow quantity and pH 5±0.5. The liquid media are filtered after 7 days for separating the cultivated mycelium, and were dried for 2 hours at 80° C. temperature for obtaining the dried mycelium.

TABLE 1
Components of Liquid Medium for Cultivating Mycelium (unit: g)

| Medium | No. 1 | No. 2 | No. 3 | No. 4 | No. 5 | No. 6 | No. 7 |
|---|---|---|---|---|---|---|---|
| Sugar | 2 | | | 1 | | | 2 |
| Malt Extract | | 2 | | 0.3 | | | |
| Potato | 20 | 20 | | | | | |
| Enzyme Extract | | | 0.2 | | | 0.3 | |
| Peptone | | | 0.2 | | 0.5 | | |
| MgSO•7H₂O | | | 0.05 | 0.015 | 0.05 | | |
| KH₂•PO₄, | | | 0.046 | 0.015 | 0.1 | | |
| K₂•HPO₄ | | | 0.1 | | | | |
| Dextrose | | | 2 | | 1 | 2 | |
| CaCl₂ | | | | 0.005 | | | |
| NaCl | | | | 0.00025 | | | |
| FeCl₃(1%)(M [symbol] ) | | | | 0.12 | | | |
| T_amine-HCl | | | | 0.000001 | | | |
| Ebios | | | | | | 0.5 | 0.5 |
| Hyponex | | | | | | 0.3 | |
| Water | 100 M [symbol] | 100 M [symbol] | 100 M [symbol] | 100 M [symbol] | 100 M [symbol] | 100 M [symbol] | 100 M [symbol] |

The amount of each dried mycelium cultivated at each medium is following.

TABLE 2
Dried Mycelium Cultivated at Liquid Media (g/20 Ml)

| Medium | C. militaris | C. nutans |
|---|---|---|
| No. 1 | 0.196 | 0.198 |
| No. 2 | 0.134 | 0.128 |
| No. 3 | 0.262 | 0.262 |
| No. 4 | 0.092 | 0.102 |
| No. 5 | 0.164 | 0.172 |
| No. 6 | 0.560 | 0.342 |
| No. 7 | 0.120 | 0.230 |

2) Cultivating Fermented Mycelium at Solid Media

At the solid media, the mycelium may be obtained from a mycelium cultivated at a liquid medium, and, for example, the mycelium cultivated at the liquid medium No.3 may be inoculated at the solid media evenly for obtaining a mycelium. The solid media was maintained 70 to 90% relative humidity and 20 to 30° C. temperature, and were cultivated for 25 to 40 days.

TABLE 3
Formation of Solid Media for Cultivating Mycelium
Formation of Solid Media

Rice 5 g + Liquid Medium 50 M [symbol]
Potato 5 g + Liquid Medium 50 M [symbol]
Corn 5 g + Liquid Medium 50 M [symbol]
Rice 5 g + Corn 5 g + Liquid Medium 50 M [symbol]
Rice 50 g + Silkworm Pupa 5 g + Liquid Medium 75 M

[symbol]

3) Cultivating Mycelium at Worm Body

The condition for cultivating at the worm bodies was equal to or same as that of at the solid medium.

TABLE 4
Formation of Worm Body Media for Cultivating Mycelium
Formation of Worm Body Medium

Silkworm Pupa 20 g + Sterilized Water 100 M [symbol]
Silkworm Pupa 20 g
Silkworm Pupa 20 g + Clean Cool Water 20 M

[symbol]

The cultivating state at the liquid media, the solid media and the worm body media was checked, and it was difficult for the mycelium cultivated at the solid media to be separated completely, hence the result of cultivation at the solid media was observed in naked eye for measuring the mycelium growth area ratio per medium area.

TABLE 5
Vegetable Worms Mycelium Cultivated at Solid Media

| Solid Media | Mycelium Growth |
|---|---|
| Rice 5 g + Liquid Medium 50 M [symbol] | + |
| Potato 5 g + Liquid Medium 50 M [symbol] | ++ |
| Corn 5 g + Liquid Medium 50 M [symbol] | +++ |
| Rice 5 g + Corn 5 g + Liquid Medium 50 M [symbol] | +++ |
| Rice 50 g + Silkworm Pupa 5 g + Liquid Medium | +++ |
| 75 M [symbol] | |

TABLE 6
Vegetable Worms Mycelium Cultivating at Worm Body

| Worm Body Medium | Mycelium Growth |
|---|---|
| Silkworm Pupa 20 g + Sterilized Water 100 M [symbol] | +++ |
| Silkworm Pupa 20 g | ++ |
| Silkworm Pupa 20 g + Clean Cool Water 20 M [symbol] | +++ |

+: 10 to 30% Mycelium Formation
++: 30 to 70% Mycelium Formation
+++: Above 70% Mycelium Formation

As shown in Table 3 to Table 6, the mycelium was cultivated very well at the solid medium of Corn 5 g+Liquid Medium 50 M [symbol] , and was cultivated well at solid medium of rice 5 g+corn 5 g+liquid medium 50 M [symbol] . And also it was found that in case of the worm body medium, the growth of the mycelium is best at the medium of silkworm pupa 20 g+Sterilized Water 100 M [symbol] .

2. Cultivation of Fruit Body by Fermentation

1) Formation of Fruit Body at Solid Medium

A fruit body was cultivated with the cultivated mycelium at the solid medium, and the fruit body was cultivated at a solid medium. For the fruit body, the condition of the solid medium was maintained at 50 to 70% humidity content, 80 to 95% relative humidity and 15 to 25° C. atmosphere temperature, and 800 to 1500 lux of light was illuminated for 60 to 90 days to cultivate a fruit body through a mycelium.

TABLE 7
Formation of Fruit Body

| Solid Medium | Mycelium Growth |
|---|---|
| Rice 5 g + Liquid Medium 50 M [symbol] | + |
| Potato 5 g +Liquid Medium 50 M [symbol] | ++ |
| Corn 5 g + Liquid Medium 50 M [symbol] | +++ |
| Rice 5 g + Corn 5 g + Liquid Medium 50 M [symbol] | +++ |
| Rice 50 g +Silkworm Pupa 5 g + Liquid Medium | +++ |
| 75 M [symbol] | |

+: Formation of 10 to 30% Fruit Body
++: Formation of 30 to 70% Fruit Body
+++: Formation of Above 70% Fruit Body

It is difficult for the fruit body cultivated at the solid medium to be separated from the medium, and then the fruit body was observed with a naked eye for measuring a growth level of fruit body formation in ratio of a fruit body formation area to a cultivating area.

2) Formation of Fruit Body at Worm Body

A fruit body was cultivated at a worm body by inoculating a mycelium cultivated a solid medium evenly the worm body, and for cultivating the fruit body at the worm body, the condition of the worm body was maintained the same as that of the solid medium.

TABLE 8
Vegetable Worms Cultivated at Worm Body

| Worm Body Medium | Fruit Body Growth |
|---|---|
| Silkworm Pupa 20 g + Sterilized Water 100 M [symbol] | +++ |
| Silkworm Pupa 20 g | ++ |
| Silkworm Pupa 20 g + Clean Cool Water 20 M [symbol] | +++ |

+: Growth of 10 to 30% Fruit Body
++: Growth of 30 to 70% Fruit Body
+++: Growth of Above 70% Fruit Body

As shown in Table 7 to Table 8, the fruit body was cultivated very well at the solid medium of rice 50 g+silkworm pupa 5 g+liquid medium 75 M [symbol] , and was cultivated well in turn rice 5 g+corn 5 g+liquid medium 50 M [symbol] , rice 5 g+liquid medium 50 M [symbol] and potato 5 g+liquid medium 50 M [symbol] . In case of cultivating the fruit body at the worm body, the fruit body was cultivated very well at the medium of silkworm pupa 20 g+clean cool water 75 M [symbol] .

3. Analyzing Components of Mycelium and Fruit body

The components of the mycelium and fruit body cultivated at the liquid media, the solid medium and the worm body were analyzed. The mycelium cultivated at the liquid medium was filtered to be freeze-dried, and the obtained fruit body was freeze-dried. And then yielded mycelium and the fruit body were analyzed according to the raw protein measuring method of Micro-Kjeldahl, the raw fat measuring method of

Soxhlet Extraction Method and the raw ash measuring method of strong drying reduction method described in the Korean Food Standard Codex. And also the components of a vegetable worm in nature selected in random were analyzed as the same manner tor comparing. In case of the natural vegetable worm, it is difficult for the mycelium and the fruit body to be separated, and the total components.

TABLE 9
Comparison Cultivated Mycelium and Fruit Body of Vegetable Worms to
Those of Natural Worms (unit: %)

| Component | Mycelium | Fruit Body | Natural |
|---|---|---|---|
| Humidity | 2.8 | 6.0 | 10.84 |
| Raw Fat | 3.07 | 2.08 | 8.4 |
| Raw Protein | 40.36 | 30.93 | 25.32 |
| Raw Ash | 7.51 | 5.32 | 4.1 |

As shown in Table 9, it is found that the components of the cultivated and fermented mycelium and fruit body according to the present invention are similar as those of the natural vegetable worms. Therefore, it means that the mycelium and fruit body may be a synthetic cultivated type of vegetable worms. Hence, the mycelium and fruit body according to the present invention may be utilized as an antibiotic preserving agent or a freshness keeping agent based on the cordycepin component. Furthermore, for enhancing the effect of cordycepin extracted from the vegetable worms cultivated according to the present invention, the vegetable worms can be fermented, or an additive such as green tea, garlic, coconut or grape can be added in the course of extraction. Or for improving the capability of antibiotic or freshness keeping property, a natural polymer may be added in the course of extraction.

A test for verifying the antibiotic and the freshness keeping effect of the mycelium or fruit body according to the present invention was performed.

4. Effect Test

An antibiotic preserving agent and a freshness keeping agents were prepared using the extracted mycelium and fruit body for testing the effect, and the agents were classified as BHC-G3 that has no acetic acid component and BHC-F3 that has acetic acid component. And antibiotic activity, anti-oxidative activity (DPPH), MIC, bio-gradable activity, sensing evaluation or preserving capability.

1) Antibiotic Preserving Test

Hamburger Patties, fish cakes and cool noodles were chosen for the antibiotic preserving effect test of the cultivated mycelium and fruit body according to the present. And a storing capability test was performed as an accelerated storing test at 12° C. temperature for choosing an antibiotic preserving agent type and concentration level.

(1) Antibiotic Preserving Effect

A reference According to MIC (Minimum Inhibitory Concentration) result was set to the hamburger patties, fish cakes and cool noodles, and tests for 1/2×MIC and 2×MIC concentration were performed.
Hamburger patty: 0.200, 0.400 and 0.800 wt %
Fish cake: 0.225, 0.450 and 0.900 wt %
Cool noodle: 0.100, 0.200 and 0.400 wt %

BHC-G3 and BHC-F3 were mixed with 10 M [symbol] water to be the above-mentioned concentrations, the hamburger patties, fish cakes and noodles were treated to be made in 10 g, respectively. And then each specimen was input into zipper-bags to store at 12° C. for 1, 2, 4 and 6 days to observe the results. The microbiological quality analyzing, pH measuring, suitable acidity measuring and sensing test were performed, and the result of tests was analyzed using the ANOVA procedure of SAS statistical program (Version 9.1, SAS Institute, Cary, N.C., U.S.A). If each treated specimen represents some statistical considerable difference (p≤0.05), the mean value was evaluated using Duncan's multiple range test.

(2) Analyzing Biological Quality Property

For the total number of bacteria test, each 10 g specimen was chosen from the sterilized bag (3M) to be mixed with a sterilized 0.2% peptone water (PW, Difco, Detroit, Mich., U.S.A) according to Rojas-Graua et al. (2008), and then the resultants were stirred for 90 second using Stomacher (Bag-mixer 400, Inter-science, France). Each 0.1 mL specimen was poured to be diluted with 9.9 mL diluting solution in stages. Each distilled specimen 1 mL was poured at 3M™ aerobic count Petrifilm (3M, St Paul, Minn., U.S.A) for cultivating at 37° C. for 24-48 hours, and then the number of colony was counted to label as log CFU/g. For measuring the number of yeast/mold, each specimen was diluted in stages after pre-treating to be poured each dried medium film (for example, 3M Yeast and mold petrifilm) by 1 mL, and then was cultivated at 25° C. temperature for 3 to 5 days to count the number of colonies in similar manner of the total number bacteria. For measuring the number of colon bacteria/colon bacterium colonies, each specimen was each specimen was diluted in stages after pre-treating to be poured each dried medium film (for example, 3M Yeast and mold petrifilm) by 1 mL, and then was cultivated at 37° C. temperature for 24 to 48 hours to count the number of colonies in similar manner of the total number bacteria.

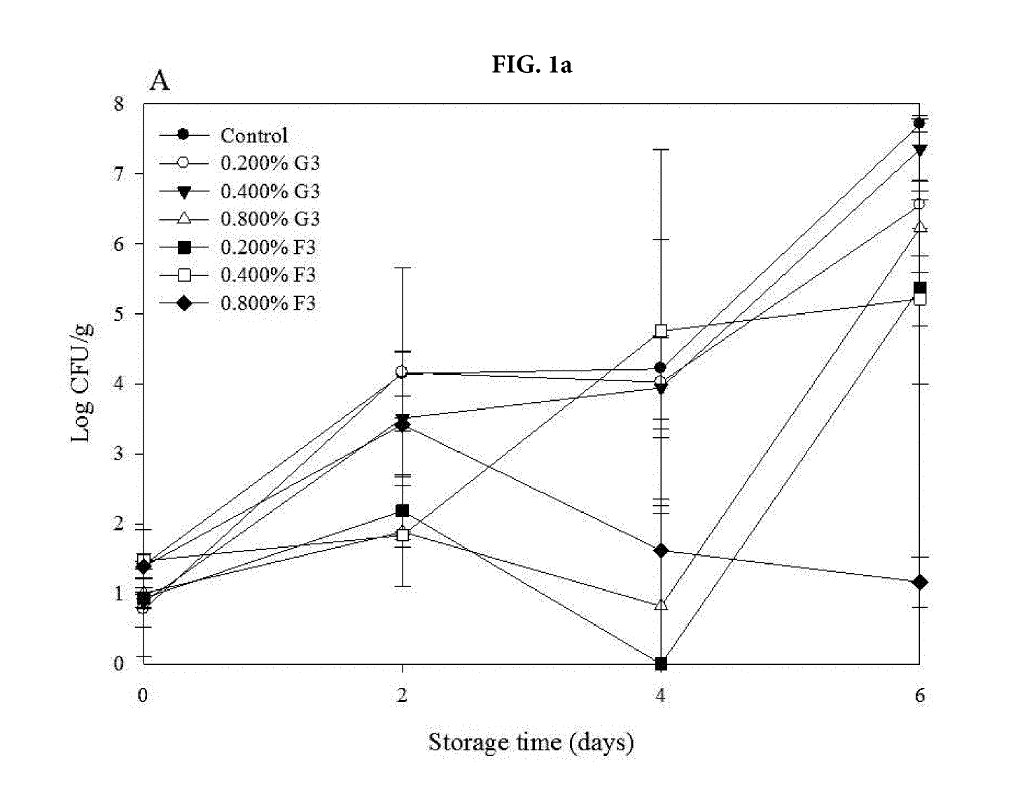

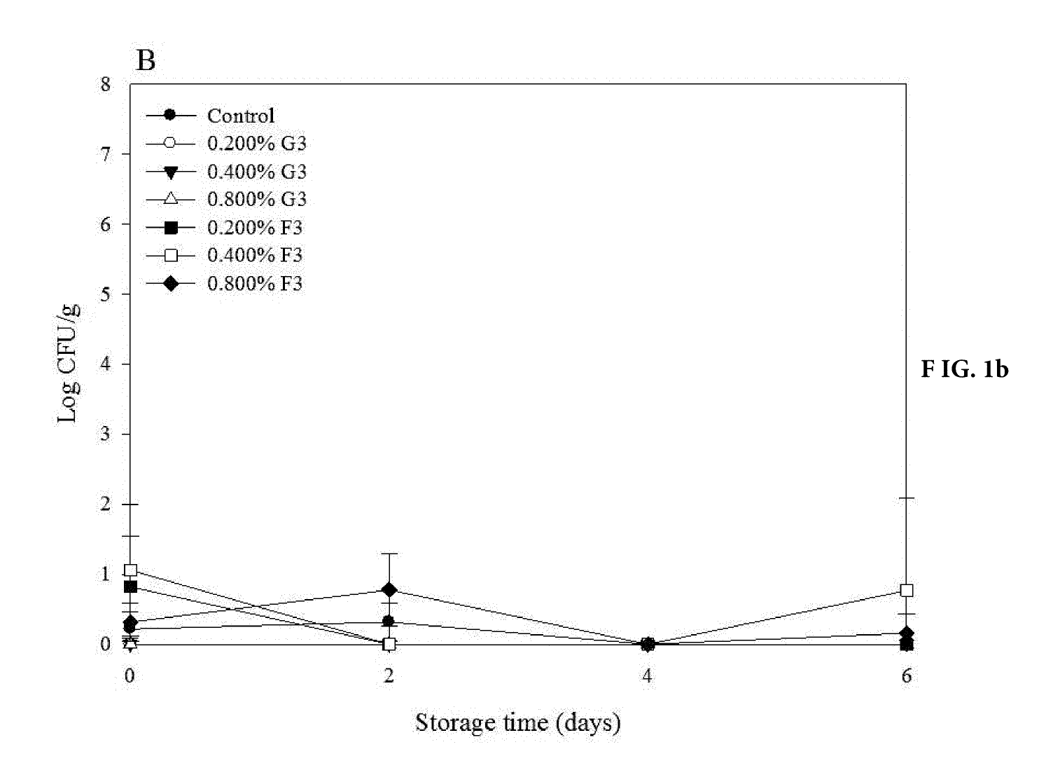

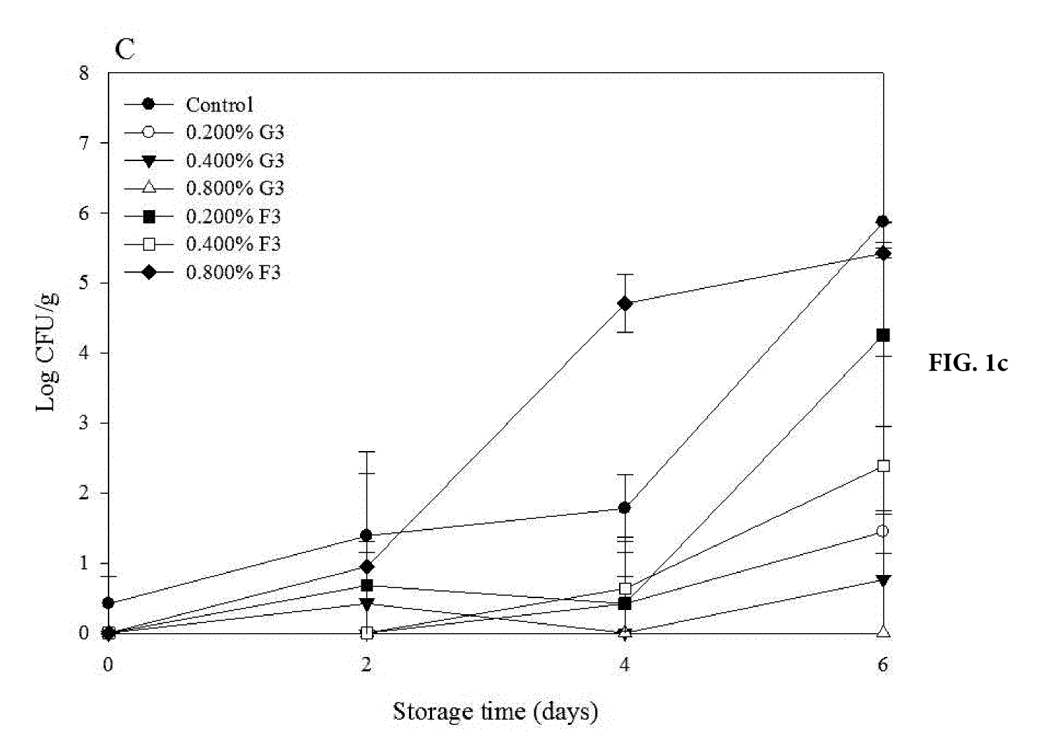

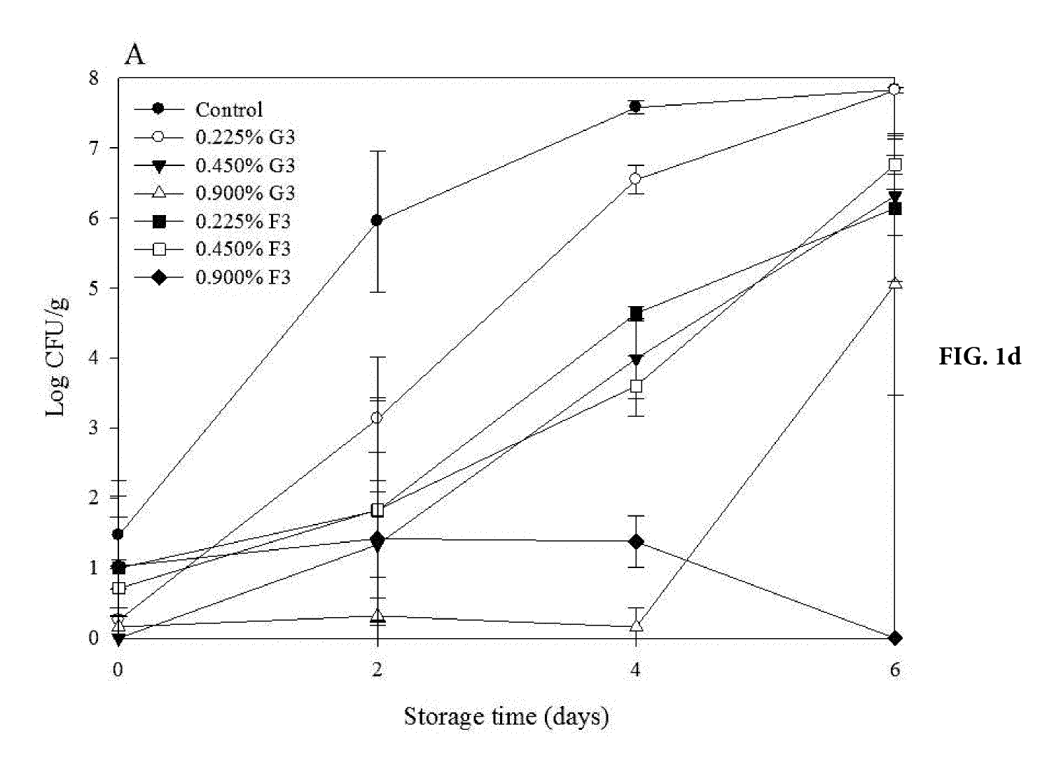

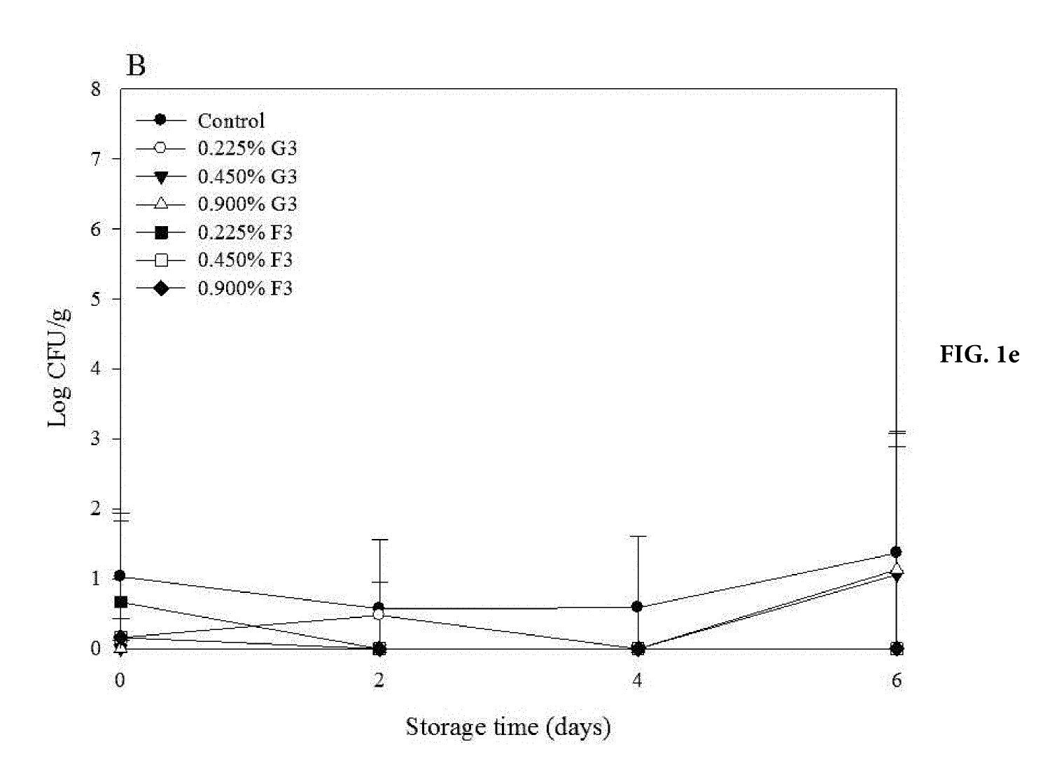

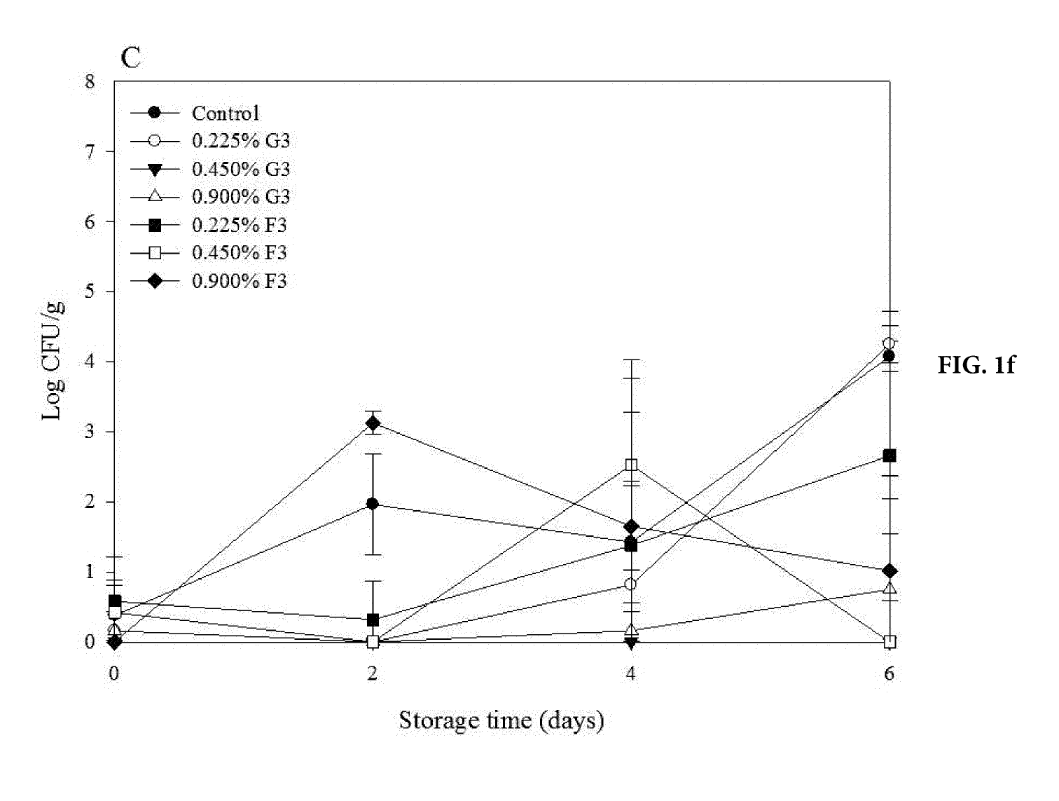

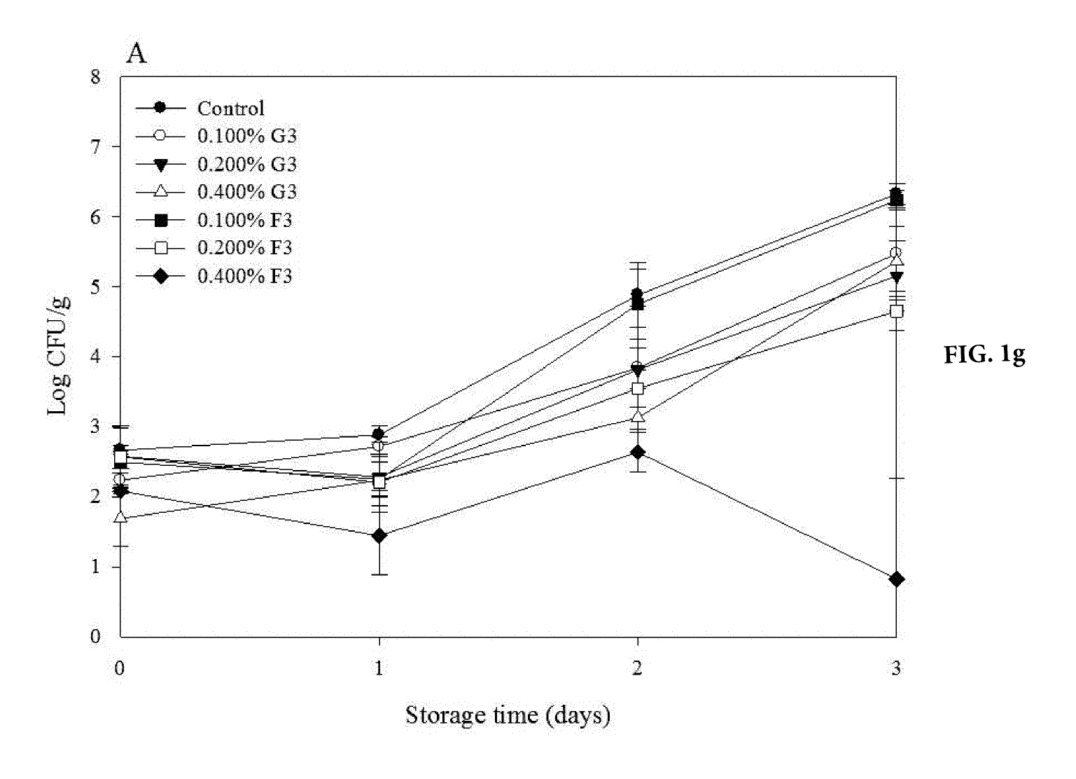

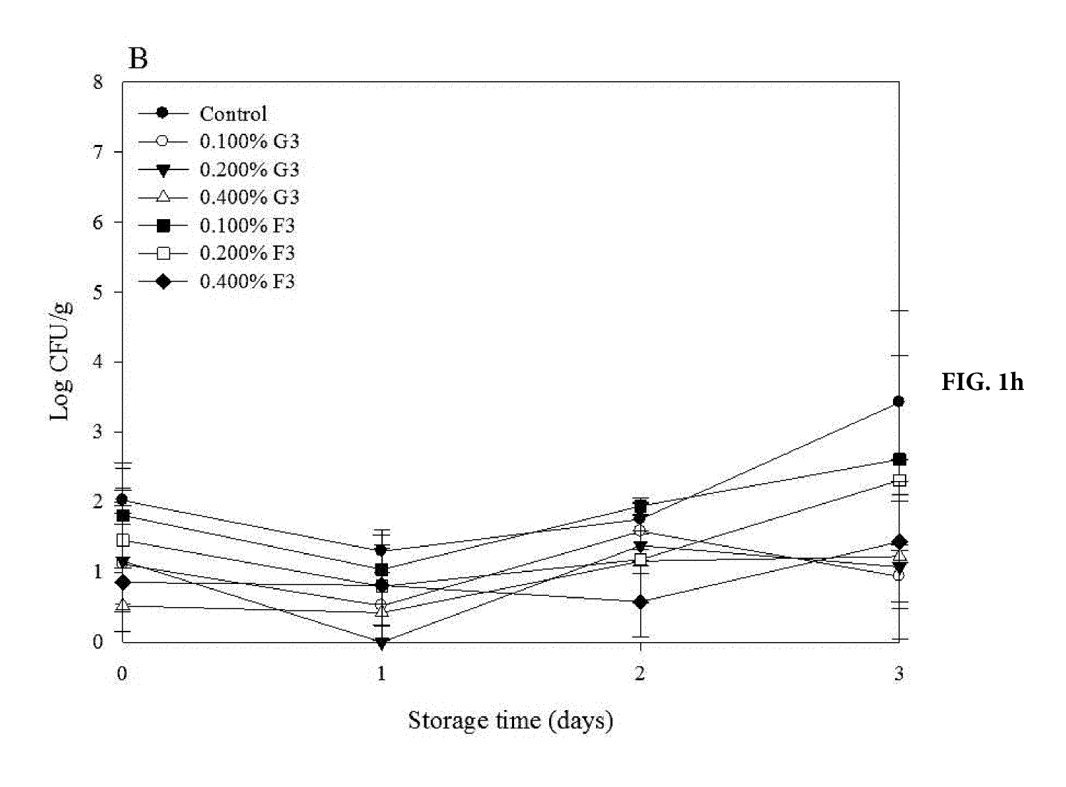

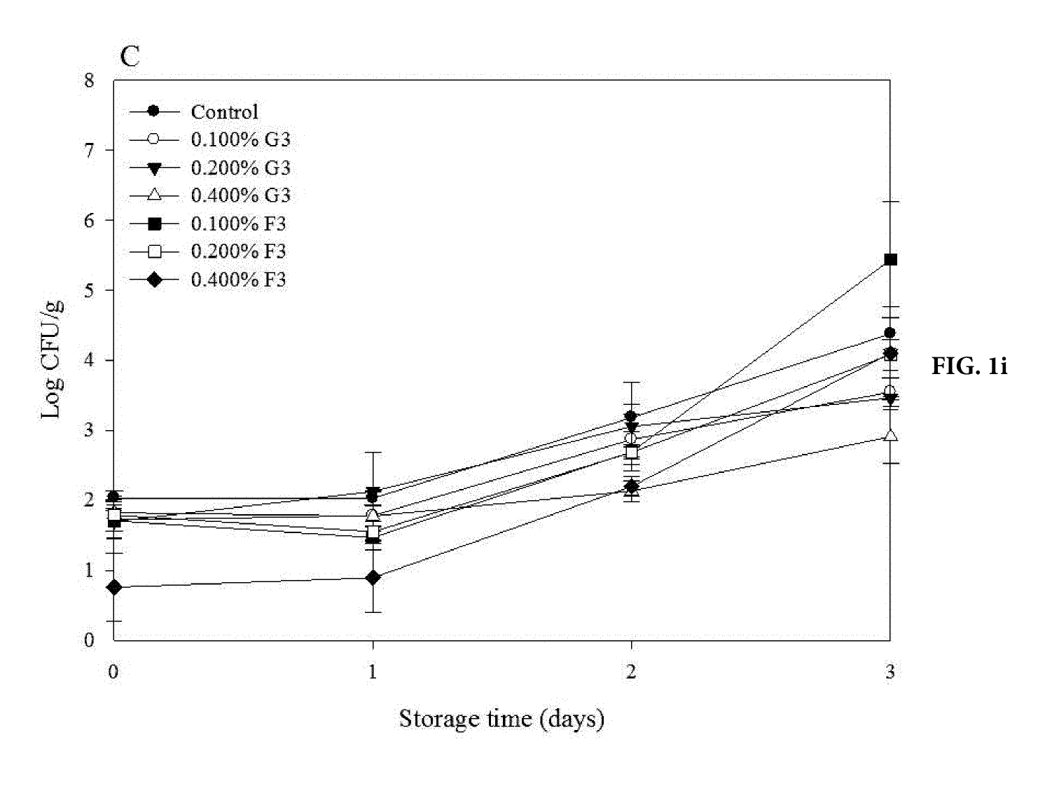

The results are shown in FIG. 1a to FIG. 1i.

FIG. 1a to FIG. 1c represents the number of the total bacteria, colon bacteria/colon bacterium colonies and yeast/molds when the antibiotic preserving agent is added. G3 and F3 were added in 0.200, 0.400 and 0.800 wt % concentration to be observed at 12° C. temperature for 0, 2, 4 and 6 days of storing period. It is found that the level of the total bacteria is lower than the control up to 0.83 to 1.89 log CFU/g when 0.800 wt % G3 is added to be store for 4 days. In particular, on adding 0.800 wt % G3, the number of yeast/mold is reduced up to the detection limit (≤0.48 log CFU/g) for 6 days of storing period. When 0.800 wt % F3 is added, the inhibiting effect of the total bacteria is the highest up to 6.54 log CFU/g for 6 days of storing period. But on adding 0.800 wt % F3, the inhibiting effect of yeast/mold is rapidly lower in forth day since storing even though the effect maintains for 2 days of storing period. Therefore, in case of hamburger patties, the inhibiting effect is the highest in view of biological safety for 4 days of storing period, when 0.800 wt % G3 is added.

FIG. 1d to FIG. 1f shows the number of the total bacteria, colon bacteria/colon bacterium colonies and yeast/molds when the antibiotic preserving agent according to the present invention to each fish cake specimen. G3 and F3 were added in 0.225, 0.450 and 0.900 wt % concentration to store at 12° C. temperature for 0, 2, 4 and 6 days, and then the result was checked. When 0.900 wt % G3 was added, the number of the total bacteria was inhibited in lower degree of 7.42 log CFU/g compared with the control in 4 days of the storing days. In sixth days of the storing days, the number of the total bacteria increased rapidly, but was maintained lower in 2.78 log CFU/g compared with the control. When 0.900 wt % F3 was added, the number of total bacteria decreased in a degree of detection limit (≤0.48 log CFU/g) in 6 days of the storing period. The number of colon bacteria/colon bacterium colonies maintained in lower degree compared with the control, but the difference may be little (p>0.05). The number of yeast/molds maintained in lower degree of 3-4 log CFU/g compared with the control in 6 days of the storing period, when 0.450 wt % G3 and 0.900 wt % F3 were added. As a result, it is found that the most effective amount is 0.450 wt % G3 or 0.900 wt % F3 in view of biological safety.

FIG. 1g to FIG. 1i shows the number of the total bacteria, colon bacteria/colon bacterium colonies and yeast/molds when the antibiotic preserving agent according to the present invention to each noodle specimen. G3 and F3 were added in 0.100, 0.200 and 0.400 wt % concentration to store at 12° C. temperature for 0, 1, 2 and 3 days, and then the result was checked. When 0.400 wt % F3 was added, the number of the total bacteria and colon bacteria/colon bacteria colonies were inhibited in lower degree of 5.50 and 1.99 log CFU/g compared with the control in 3 days of the storing days. On being treated with G3, the number of colon bacteria/colon bacteria colonies was maintained in lower degree than the control, but the number of the total bacterial made a little difference between G3 and the control. Therefore, in case of the noodle, it is found that the most effective amount is 0.400 wt % F3 in view of biological safety.

(3) Measuring pH

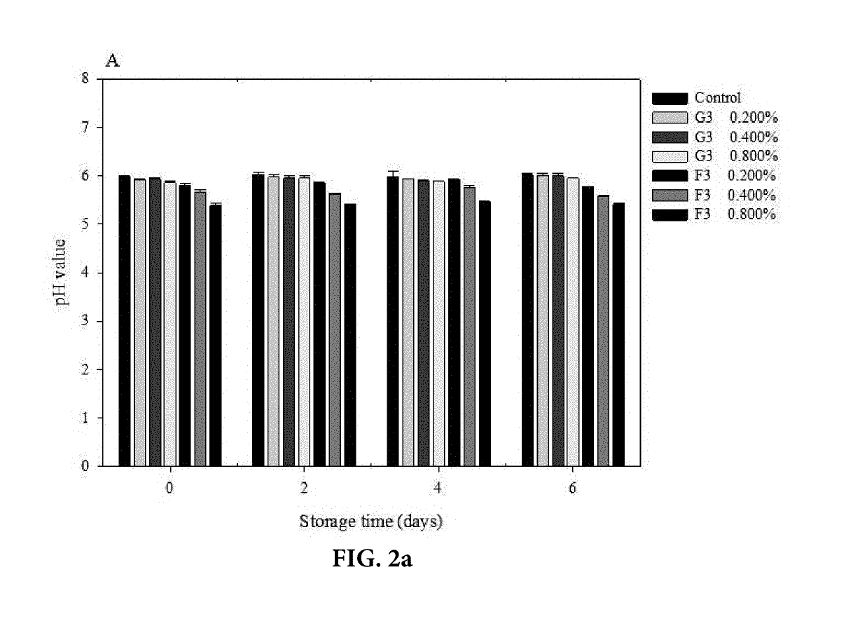

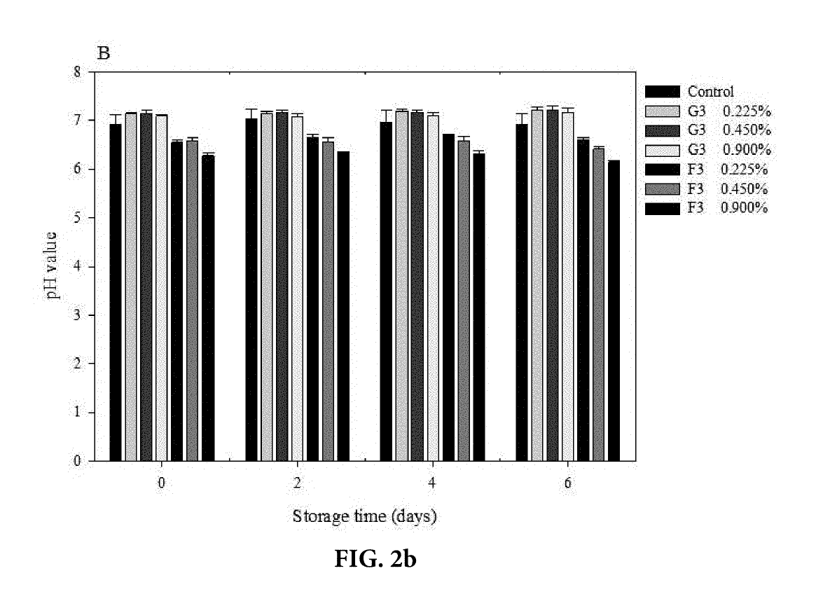

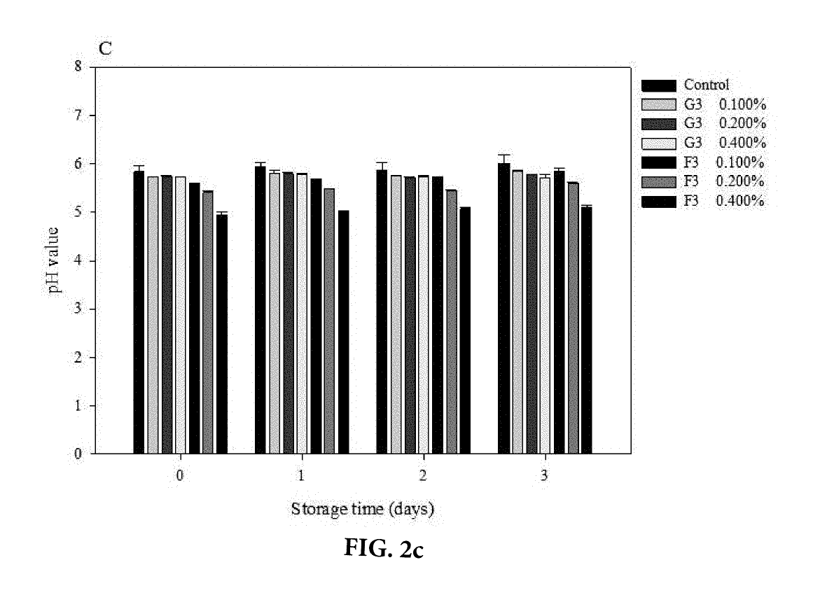

For measuring pH change of food on treating the extraction according to the present invention, each specimen was homogenized with distilled water 20 mL for 90 seconds in a sterilized bag by a stomacher according to Kwak et al. And the pH of each specimen was measured by a digital pH-meter (Beckman Counter, model pH, U.S.A). The result is shown in FIG. 2a to FIG. 2c.

Referring to FIG. 2a to FIG. 2c, the initial pH values of the hamburger patty, the fish cakes and it was found that the noodle were 5.98, 6.92 and 5.84, and G3 made no pH change in all of the concentration. On being treated with F3 2×MIC concentration, the hamburger patty, each pH value of the fish cake and the noodle decreased by 0.07, 0.78 and 0.90, but it may be said that no the substantial change happened. Therefore, in summary, no substantial change occurred in the storing period, hence it is found that G3 and F3 has no effect of the change of food.

(4) Measuring Proper Acidity

The acidity was measured according to AOAC standard test. For measuring, specimen 10 g and distilled water 10 mL were input into a sterilized bag to be homogenized for 90 seconds with a stomacher. Then one or two drops of 1% phenolphthalein solution (77-09-8, Duksan pure chemicals, Gyeonggi-do, Korea) were added to the homogenized specimen to be titrated as 0.1 N NaOH (S0610,

Samchun pure chemical, Gyeonggi-do, Korea). The point that a red state maintained for 30 second from colorlessness was considered ad a terminal point, and the amount of organic acid was calculated using the amount of the consumed 0.1 N NaOH as following.

$$Formulation 1$$

$$\% acid ( TA ) = \frac{Vol . of NaOH ( ml ) \times N ( NaOH ) \times Acid meq . factor \times 100}{Mass of sample ( g )}$$

$$Vol . of NaOH ( ml ) = Total volume of NaOH used for titration$$

$$N = Normality of titration ( NaOH )$$

$$Acid meq factor = miliequivalent weight of acid$$

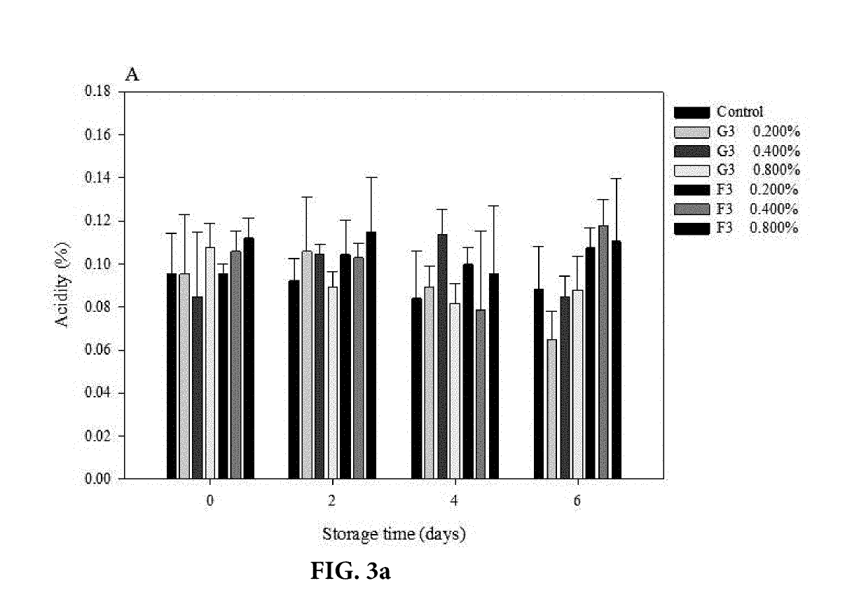

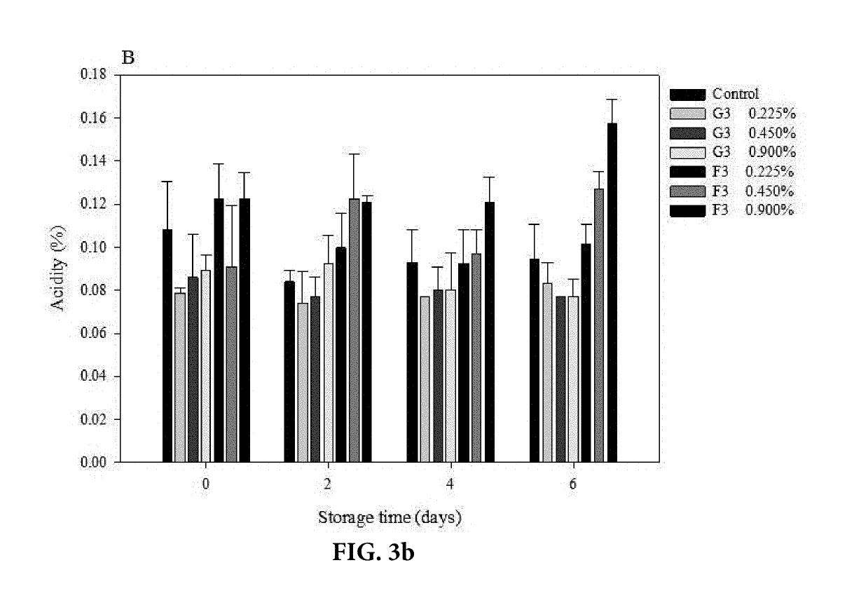

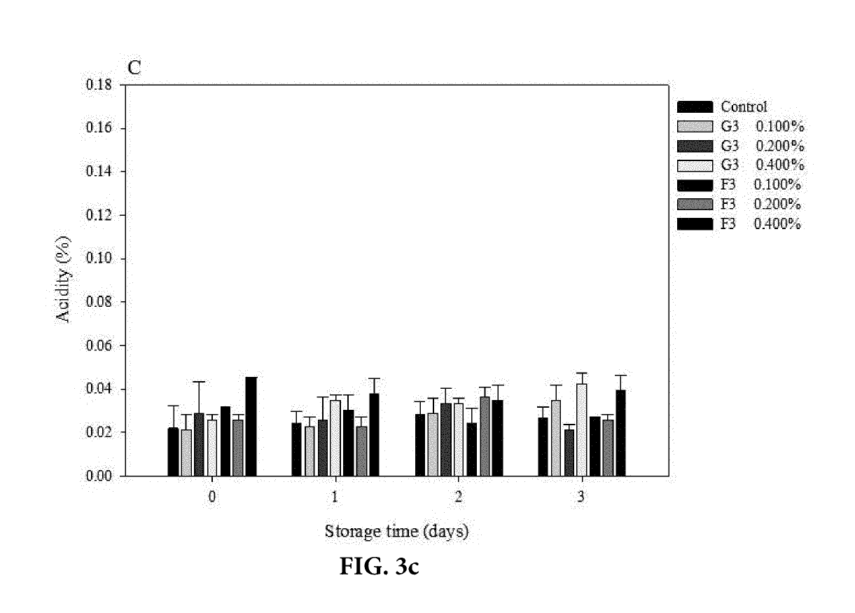

The measuring result is shown in FIG. 3a to FIG. 3c. Referring to FIG. 3a to FIG. 3c, the initial acidity of the hamburger patty, fish cake and noodle was 0.10, 0.11 and 0.02%, respectively when the antibiotic preserving agent of the present invention was added. It was found that no substantial change of acidity occurred in all of the specimens. Therefore, it is assured that the acidity of the hamburger patty, the fish cake and the noodle are not changed substantially on adding the above-mentioned concentration of G3 and F3.

2) Sensing Test

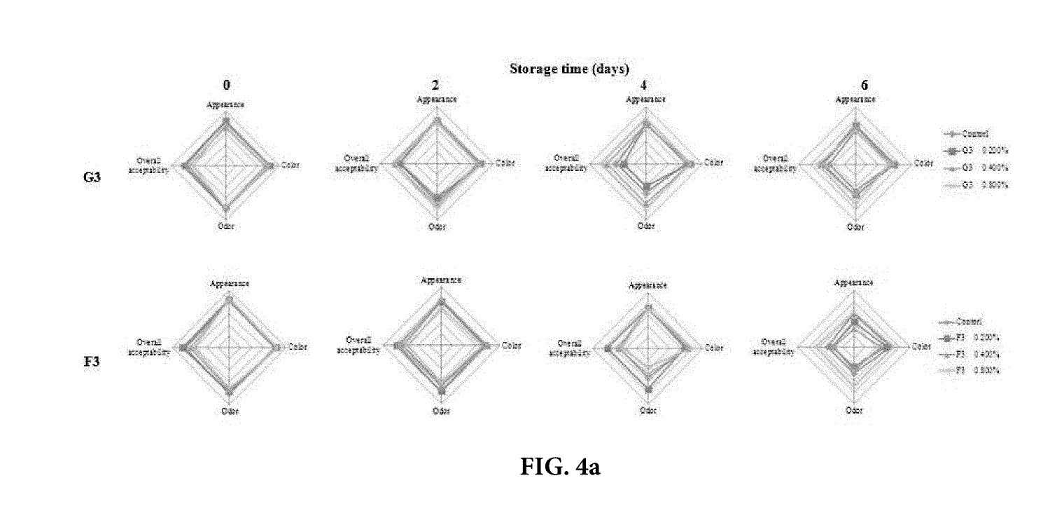

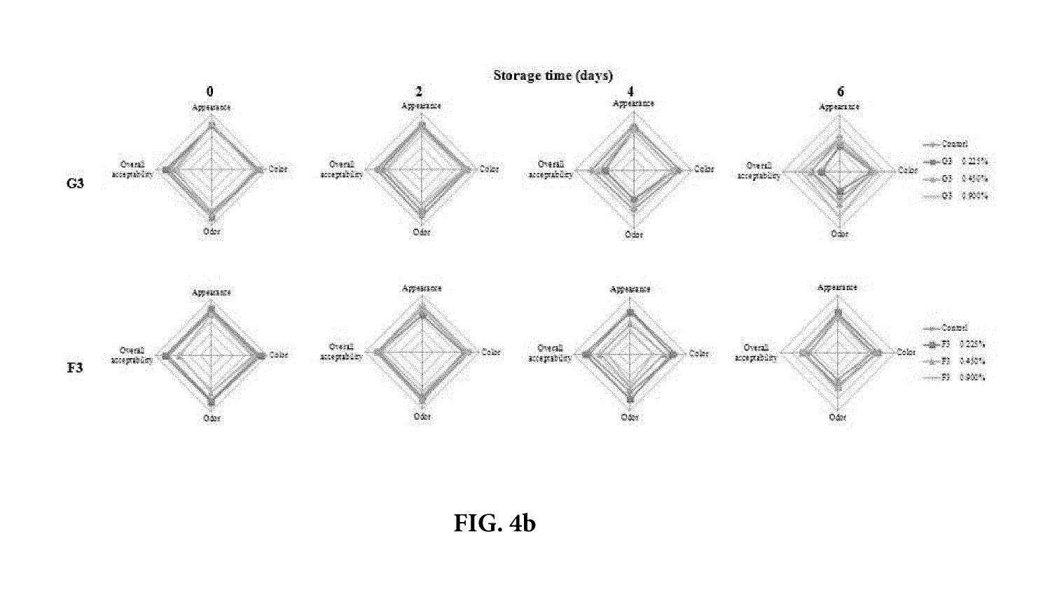

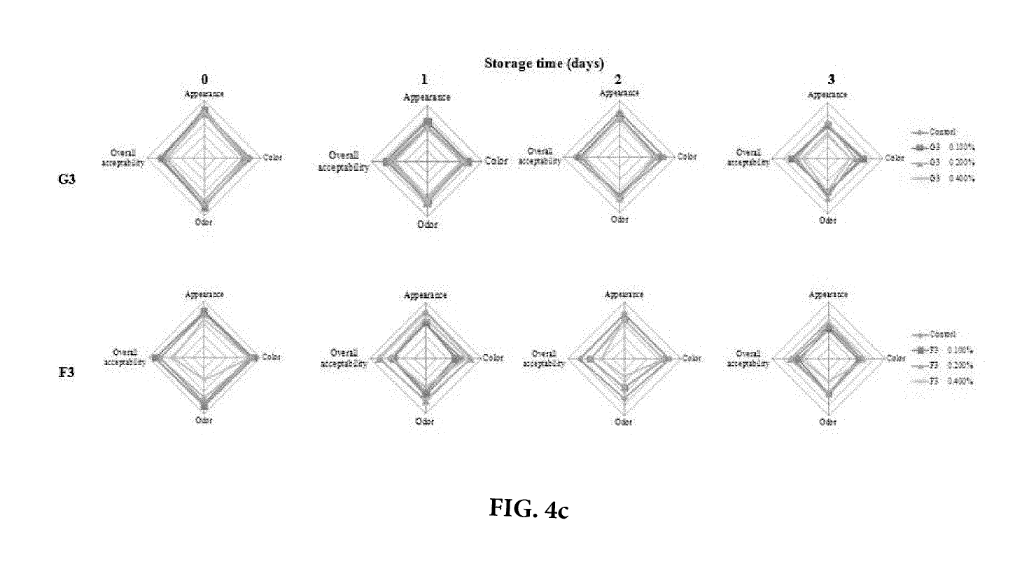

Sensing test such as appearance, color, flavor and total preference was performed with the hamburger patty, the fish cake and the noodle to one hundred subjects using 5 point scale, wherein the higher grade represents the better effect. And the taste evaluation was added for 0 day. The result is shown in FIG. 4a to FIG. 4c.

FIG. 4a shows the result of the sensing test when the antibiotic preserving agent was added to the hamburger patty. If the amount of 0.800 wt % G3 and F3 was added to the hamburger patty, it was found that the effect to the taste was not good as 1.75 and 2.50 points. On the contrary, the appearance, the color, the flavor and the total preference are acceptable as above 3.00 points at all the treated specimens. In regard to 6 days of the storing period, all of the evaluation items were shown as higher points than the control in sensing effect when treated as 0.800 wt % F3. And also, all of the evaluation items were shown as acceptable, representing the degree of above 3.0 points, if treated as 0.800 wt % G3.

FIG. 4b shows the result of the sensing test when the antibiotic preserving agent was added to the fish cake specimens. If the amount of 0.900 wt % G3, 0.450 and 0.800 wt % F3 was added to the fish cakes, it was found that the effect to the taste was not good as 2.00, 2.67 and 2.50 point. On the contrary, the appearance, the color, the flavor and the total preference are acceptable as above 3.00 points at all the treated specimens. But it was found that the appearance, the color and the flavor were shown as acceptable. On the contrary, in regard to the total preference, all of the evaluation values were shown as 2.86 points in a lower degree, when 0.450 and 0.900 wt % F3 were added. A higher point was shown for the storing period than other specimens, when treated as 0.900 wt % G3. A point equal to or higher was shown for 6 days of the storing period than the control, representing the treatment effective to the sensing.

FIG. 4c shows the result of the sensing test when the antibiotic preserving agent was added to the noodle specimens. In regard to 0 day, the evaluation points were shown above 3.00 points as acceptable. And also, the appearance and the color were shown in 3.43 to 4.29 points as acceptable, even though the point was lower than the control in the range of a little difference. But for 0 day of the storing period, the flavor and the total preference were shown in 2.00 and 2.86 as unacceptable, when treated as 0.400 wt % F3. In regard to 6 days of the storing period, all of the evaluation items were shown above 3.00 as acceptable, when treated as 0.100 wt % G3. And the appearance and the color were shown above 3.00 points as higher than the control, representing good effect to the sensing when treated as 0.200 and 0.400 wt % F3.

In summary, the sensing except the taste is not affected by the addition of 0.800 and 0.900 wt % G3 to the hamburger patty and the fish cake. In case of the noodle, 0.100 wt % G3 or 0.200 wt % F3 is not affected to the sensing.

3) Toxic Test

The toxic test was performed according to the MTT analyzing method, in which the capability of mitochondria that a yellow soluble matrix reduces MTT tetrazolium soluble into insoluble is utilized by dehydrating enzyme effect.

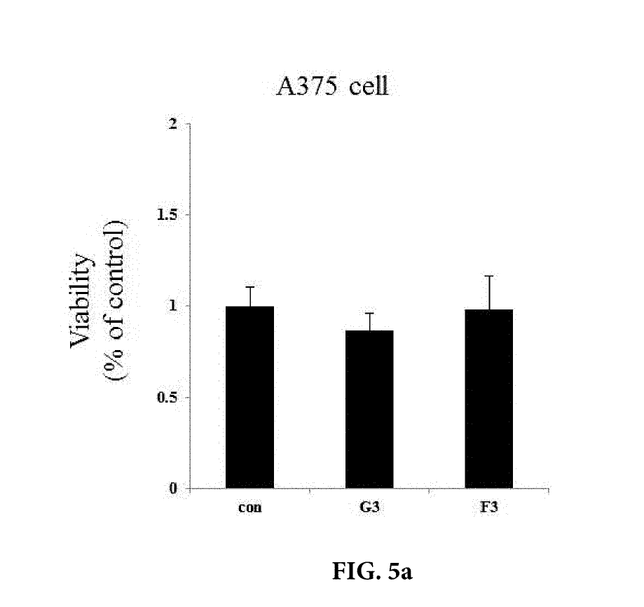

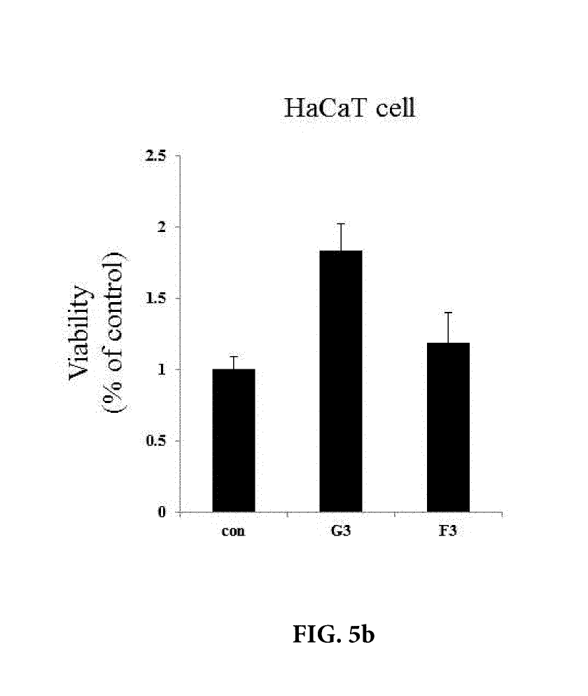

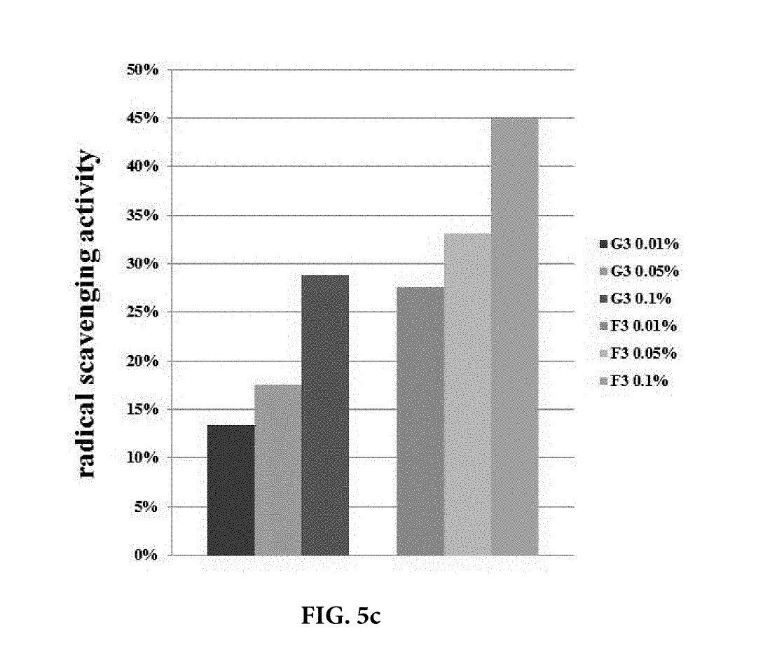

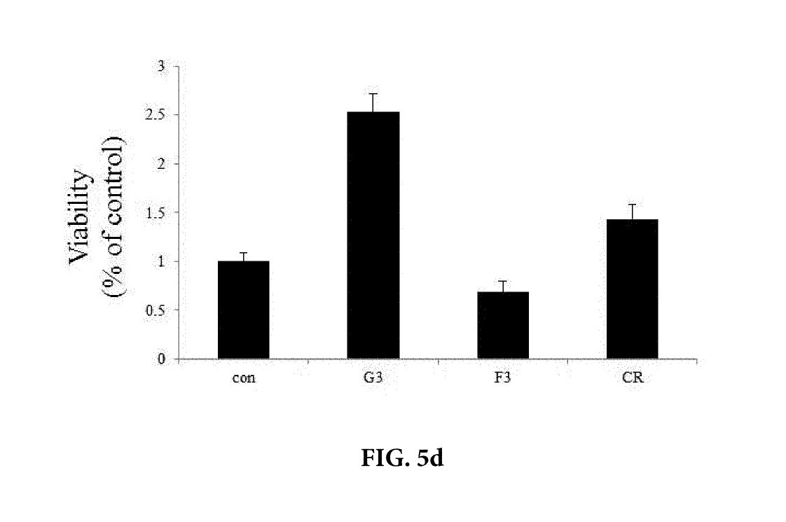

The light absorbance of untreated samples was measured, and then each sample was treated to test toxicity in a manner wherein the absorbance is measured at 540 nm using a micro-plate reader. The result is shown in FIG. 5a to FIG. 5d.

Referring to FIG. 5a to FIG. 5d, a corneous cell such as HaCat cell had a result of increasing growth when treated as G3 of 1 wt % concentration, and no toxic effect appeared. And it was found that the growth of a normal corneous cell and a cancer cell generating melanin was not affected by 1 wt % F3.

(1) Freshness Keeping Agent

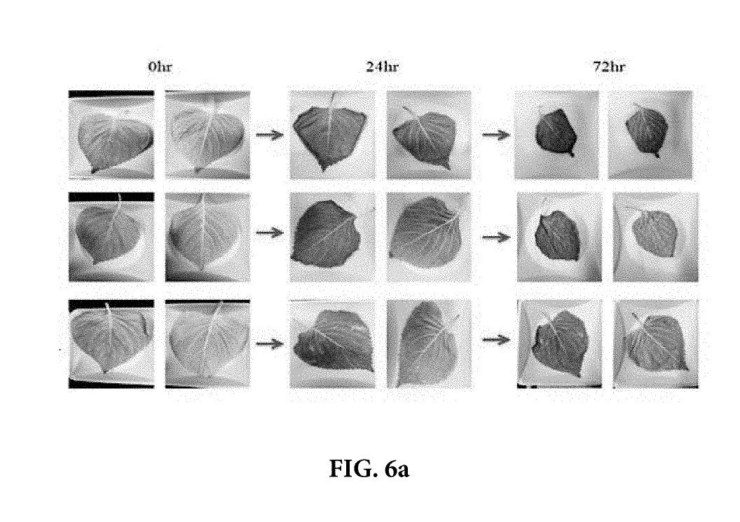

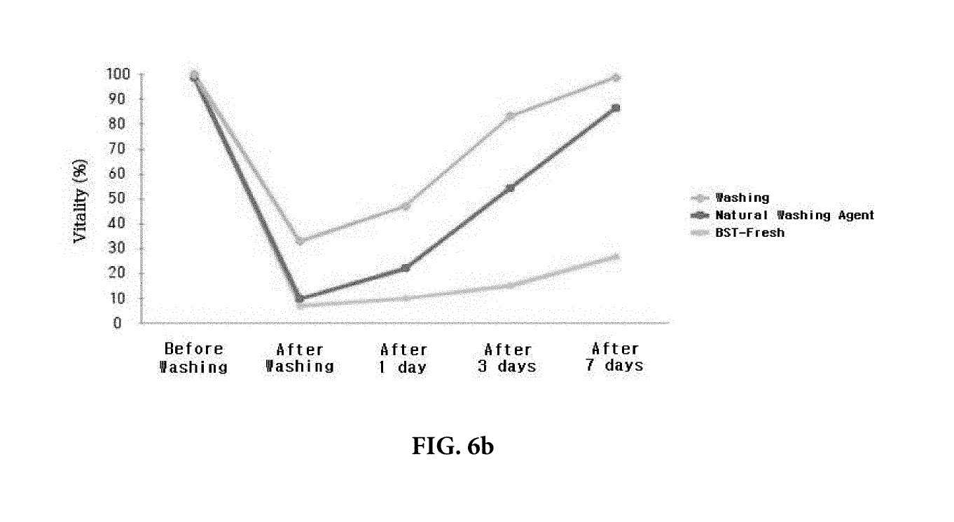

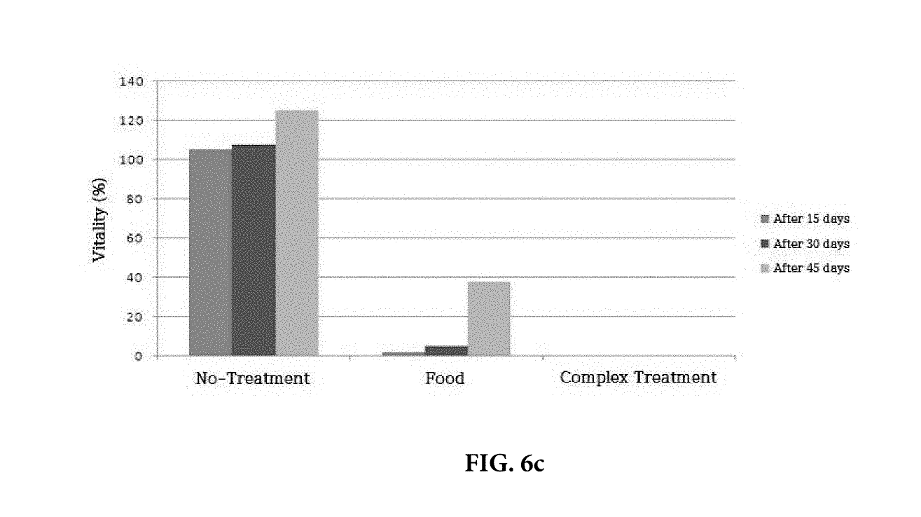

G3 and F3 were tested with green vegetables for freshness keeping capability, and the result was shown in FIG. 6a to FIG. 6c.

Referring to FIG. 6a to FIG. 6c, it is advantageous that the vegetables are treated with a natural preserving agent after being treated with the composition according to the present invention for inhibiting bacteria. In particular, if the vegetables are treated with G3 or F3 for freshness keeping capability to be processed, the effect of the natural preserving agent may increase.

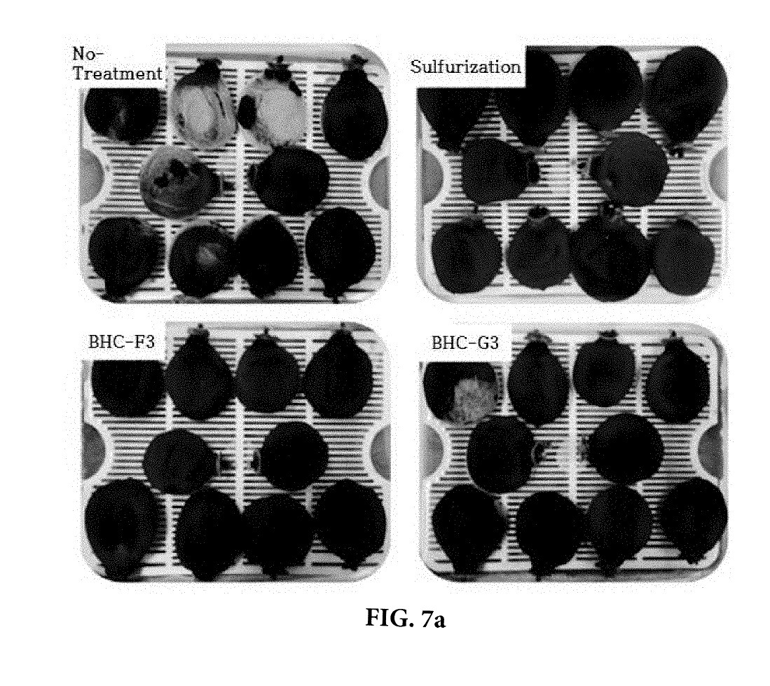

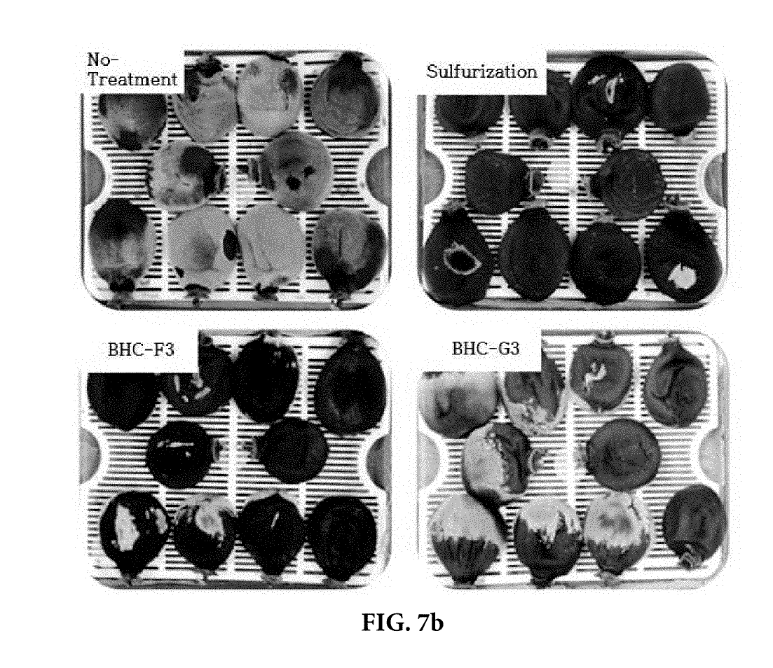

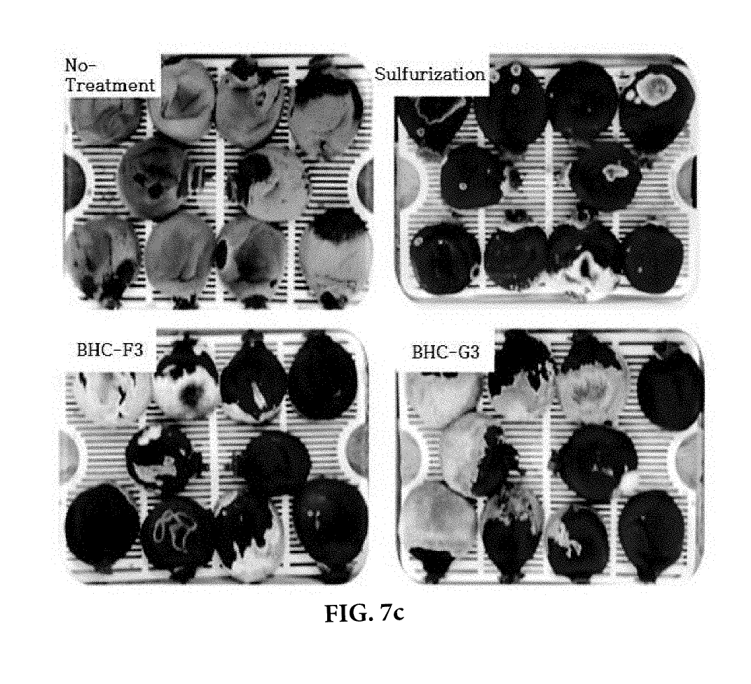

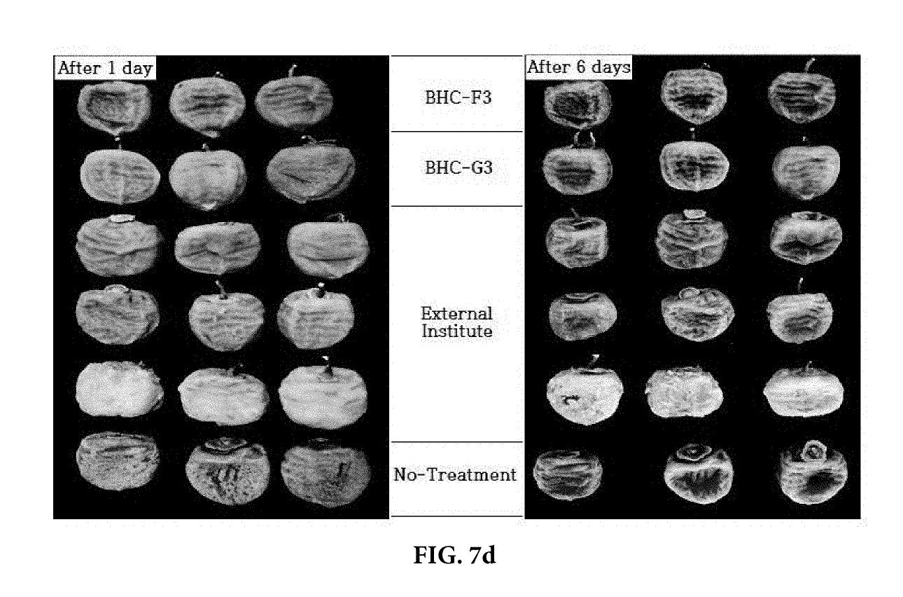

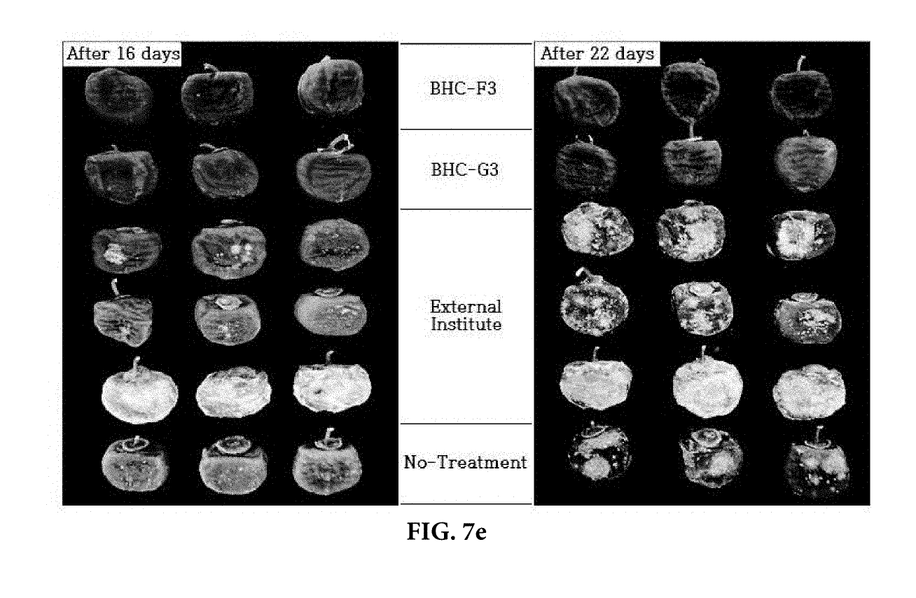

For testing the effect of the freshness keeping agent, a solution that the concentration of G3 or F3 was 3 to 6% was prepared to be dried after immersing into the solution for 50 to 80 seconds. And then, a spore was obtained by adding distilled water to a flat medium wherein two kinds of fungi separated from the dried persimmon and one kind of possessing strain was cultivated. And the bacteria number of the obtained fungus spores was checked through a microscope or smearing, the fungus spores were diluted with the concentration controlled, and then they were inoculated at the dried persimmons in many times. And then the condition of adding humidity was maintained after inoculating to be input for observation. And the result is shown FIG. 7a to FIG. 7e.

FIG. 7a to FIG. 7e shows the observation result in 5 days, 10 days and 20 days, respectively, FIG. 7d and FIG. 7e show the observation result in 1 to 6 days and 22 days by the external professional institute.

Referring to FIG. 7a to FIG. 7e, under the cruel condition such the fungus inoculation and humidity addition, the fungus was developed before 5 days in case of no treatment, and the fungus was developed little in case of sulfurization, BHC-F3 and BHC-G3. Specifically, the result is as follows.

Number of Occurring Population: 8/10, Area Ratio of Fungus Occurrence: 40% of Total Area and Occurring Population Number of BHC-G3: 1/10.

And the result is in case of in 20 days of Inoculation of Fungus with Sulfurization and BHC-F3.

Occurrence Population Number: 10/10, Area Ratio of Occurrence: 20% and Occurrence Population Number: 8/10, Area Ratio of Occurrence: 40%.

As a result, under hard conditions, BHC-F3 may be substituted for the sulfurization. And also, it was found that above 80% of the participants feels no difference between no-treatment and BHC-F3 treatment, and has no feeling in case of intake alone.

While the present invention has been particularly shown and described with reference to exemplary embodiments thereof, it will be understood by those skilled in the art that various changes in form and details may be made therein without departing from the spirit and scope of the present invention as defined by the appended claims.

What is claimed is:

1. A method for cultivating vegetable worms having natural antibiotic preserving capability and freshness keeping capability, comprising:
obtaining a mycelium by inoculating a cordyceps militaris or a cordyceps nutans to a liquid medium, a solid medium or a worm body; and
forming a fruit body from the mycelium by cultivating a solid medium or a worm body
wherein the solid medium for cultivating the fruit body is formed by a mixture of a corn material and the liquid medium or a mixture of a rice material, a silkworm pupa material and the liquid medium; the solid medium is under a condition of 50 to 70 wt % moisture content, 80 to 95% relative humidity, 15 to 25° C. atmosphere temperature and 700 to 1,200 lux luminous intensity; the liquid for the liquid medium contains MgSO₄.7H₂O, KH₂.PO₄, K₂.HPO₄, a yeast extract, a peptone and a dextrose or contains a yeast extract, a dextrose, an ebios and a Hyponex; the solid medium for the mycelium is formed from the liquid medium and at least one selected from a group consisting of a rice material, a potato material, a corn material and a silkworm pupa material.

2. The method of according to claim 1, wherein comprising fermenting an extract from the mycelium or the fruit body by a lactic acid bacterium.

3. A natural complex composition of vegetable worms sprayed on green vegetables or added to food for keeping freshness or preserving antibiotic property, comprising:
an extracted component cultivated in a liquid medium, a solid medium or a worm body and consisting of 2.0 to 8.0 wt % water; 2.0 to 3.5 wt % crude fat; 30 to 50 wt % raw protein; 5.0 to 8.0 wt % raw ash; and
an additive comprising a cellulose, a pectin, a carrageenan, a starch, a protein, an enzyme or a chitosan.

4. A natural complex composition of vegetable worms sprayed on green vegetables or added to food for keeping freshness or preserving antibiotic property, comprising:
a base material extracted from vegetable worms and consisting of 2.0 to 8.0 wt % water, 2.0 to 3.5 wt % raw fat, 30 to 50 wt % raw protein and 5.0 to 8.0 wt % raw ash; and additives containing a green tea, a garlic, a coconut, a grape or an oriental medicine stuff.
